(19) United States
(12) Patent Application Publication
(10) Pub. No.: US 2008/0281338 A1
(43) Pub. Date: Nov. 13, 2008

(54) Surgical Suture System

(76) Inventors: Stephen Wohlert, Norderstedt (DE); Raimo Sump, Hamburg (DE); Ingo Fritz, Hamburg (DE); Reinhard Juraschek, Leezen (DE); Jorg Holste, Norderstedt (DE); Krasimira Hristov, Hamburg (DE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/815,180

(22) PCT Filed: Jan. 17, 2006
(86) PCT No.: PCT/EP2006/000372

§ 371 Date: Apr. 1, 2008

(30) Foreign Application Priority Data
Jan. 31, 2005 (DE) 10 2005 004 318.6

(51) Int. Cl. A61B 17/06

(52) U.S. Cl. 606/148

(57) ABSTRACT

A surgical suture system (1, 21, 41) is illustrated and described. The object, namely to permit simple correction of the suture course or of an incorrect position, is achieved by a suture system (1) with a needle-like applicator (2, 22, 42) having a tip (26, 50) in its front end area, with a surgical suture material (4, 24, 44) provided with barbs (6, 7, 28, 29, 47, 48) which, on one side, preferably point in one direction of action from a reference point (8, 30, 49) and, on the other side, point in the opposite direction of action from the reference point (8, 30, 49) and are designed to block a movement of body tissue (12) away from the reference point (8, 30, 49), and with a sheath (3, 23, 43) whose front end area starts from the rear end area of the applicator (2, 22, 42) and which surrounds the surgical suture material (4, 24, 44).

The present application relates to a surgical suture system with a suture material provided with barbs.

A surgical suture material of this kind, with barbs for anchoring the suture material in tissue, is known from U.S. Pat. No. 3,123,077 and GB 1,091,282, for example. However, such a suture material has the disadvantage that it already catches in the body tissue when the suture material is not yet in the desired position. It can therefore be introduced into the body tissue only in conjunction with a cannula, which is complicated and time-consuming.

U.S. Pat. No. 6,241,747 discloses a suture material with barbs which, on one side, point in one direction of action from a reference point and, on the other side, point in the opposite direction of action from the reference point and are designed to block a movement of body tissue away from the reference point. The suture material with the barbs can be introduced into the tissue with the aid of a separate insertion device which surrounds the suture material. During insertion, the tissue is protected from the barbs of the suture material by the insertion device, so that the barbs do not come into contact with the body tissue during introduction.

A disadvantage of this suture material and of the insertion aid is that it is not possible to rectify an incorrect position of the suture material, in particular of the critical “middle area” where the two sections of barbs with different direction of action meet. Moreover, prior to introduction, the surgeon has to insert the suture material into the insertion aid, which is time-consuming and can lead to problems with regard to sterility.

Therefore, the object of the present invention is to make available a surgical suture system in which it is readily possible to correct the suture course or an incorrect position.

This object is achieved by a surgical suture system having the features of claim 1. Advantageous embodiments of the invention are set out in the dependent claims.

The surgical suture system according to the invention has a needle-like applicator having a tip in its front end area, and a surgical suture material provided with barbs. A sheath is also provided whose front end area starts from the rear end area of the applicator and which surrounds the surgical suture material. The barbs on one side preferably point in one direction of action from a reference point on the suture material and, on the other side, point in the opposite direction of action from the reference point and they are designed to block a movement of body tissue away from the reference point.

The expression “surgical suture material” in the sense of this invention is to be interpreted widely and, in addition to suture material or tissue connectors, for example surgical suture threads, it also includes flexible elongate implants, for example cords or bands.

In the suture system according to the invention, the position of the suture material can be easily corrected by moving the suture material with the sheath backwards and forwards in the body tissue, without causing any further damage to the tissue. Without the sheath, the barbs, when moved in the direction in which said barbs point, would tear fibres. In particular, the barbs cannot “saw” into the tissue during movement of the suture system according to the invention.

Since the suture material and the applicator do not alter their position relative to one another during insertion into the tissue, a suitable positioning of the critical “middle area” of the suture material can be easily reached. This permits the best possible anchoring effect and prevents the position of the suture material from changing after the operation.

The position of the critical “middle area” can be established via the position of the applicator, because the applicator is the only part of the system that the surgeon can observe directly. In particular, the applicator is visible even during the passage through the tissue.

Suture systems composed of applicator, suture material and sheath, and prepared and assembled in the manner according to the invention, save time during the operation and reduce the risk of infections, since the surgeon simply has to remove the suture system from the packaging and is able to use it directly. Moreover, there is no risk of the surgeon injuring himself when connecting applicator and suture material.

The already low risk of infections is further reduced by the fact that a sterilized suture material is contained in what is also an already sterilized application device. At no point during the operation does anything non-sterile come into contact with the sterile suture material, either inside or outside of the skin.

The suture system according to the invention does not use hollow needles to prepare for positioning the suture material. Because of its open cutting tip, a hollow needle would create a “tunnel” in the tissue. However, a puncture without a “tunnel” leads to a more intimate connection between the suture material and the tissue. In the system according to the invention, the applicator designed as a needle can be even smaller in diameter than the suture material itself. This makes it possible for the suture system to be flat and level even at the connection point between suture material, applicator and sheath. Therefore, it is possible to use needles of substantially smaller diameter which are more mobile and cause less damage in the tissue, which in turn later leads to more intimate anchoring. In addition, it is possible to configure applicator tips which cause less trauma to the tissue. This likewise leads to an improved anchoring of the suture material in the tissue.

In a preferred embodiment, the sheath comprises a flexible material, so that the sheath follows the applicator through the tissue without great resistance. This flexibility in terms of the surgical suture permits curves whose course is followed by the sheath without the barbs catching in the tissue.

Alternatively, the sheath can comprise a rigid material, and this material is also preferably bendable. In this case, the course of the suture during insertion of the suture system can also be determined from the rear area of the sheath. In this case, it is particularly preferred if the applicator and the sheath are made in one piece and a separation point is provided between applicator and sheath, which separation point is further preferably designed as a predetermined weak point. In this way, it is possible to make available a suture system in which the sheath can be easily separated from the applicator after insertion into the tissue, and the suture material can be released by removal of the sheath.

In a further preferred embodiment, the suture material is secured on the applicator. In this case, the sheath, after insertion into the tissue, can be removed counter to the direction of insertion, and the suture material can be held on the applicator and also later separated from this, if appropriate. Alternatively, however, it is also possible for the suture material not to be secured on the applicator, with the result that the latter is freely movable in relation to the suture material.

The rear end area of the sheath can either be open or closed. If it is open, the sheath can be drawn forwards out of the tissue in the direction of insertion, the suture material being held at its rear end. If the rear end area is closed, the sheath is drawn out of the tissue counter to the direction of insertion. In this case, the suture material does not have to be secured on the applicator, with the result that subsequent detachment of the applicator is not necessary, which saves time. In addition, the suture material is protected against soiling during introduction.

It is also preferred if the suture material comprises an active substance. This prevents infections and improves the uptake of the suture material in the tissue. In a further preferable embodiment, the sheath and/or the applicator can also be provided with an active substance or a lubricant, which additionally facilitates the passage of the suture system through the tissue.

In a further preferred embodiment of the invention, the transition area between applicator and sheath has an atraumatic design. This means in particular that in the transition area there are no steps or edges, so that the shape of the transition area causes only minimal damage upon insertion into the tissue, and the suture material is later optimally anchored in the tissue.

It is also preferred if a further applicator is arranged at the rear end of the sheath. This permits use of a single, continuous suture material through three punctures.

Further preferred embodiments of the invention are described in the dependent claims.

The invention is explained below with reference to a drawing which shows three illustrative embodiments of the invention which are purely preferred. In the drawing:

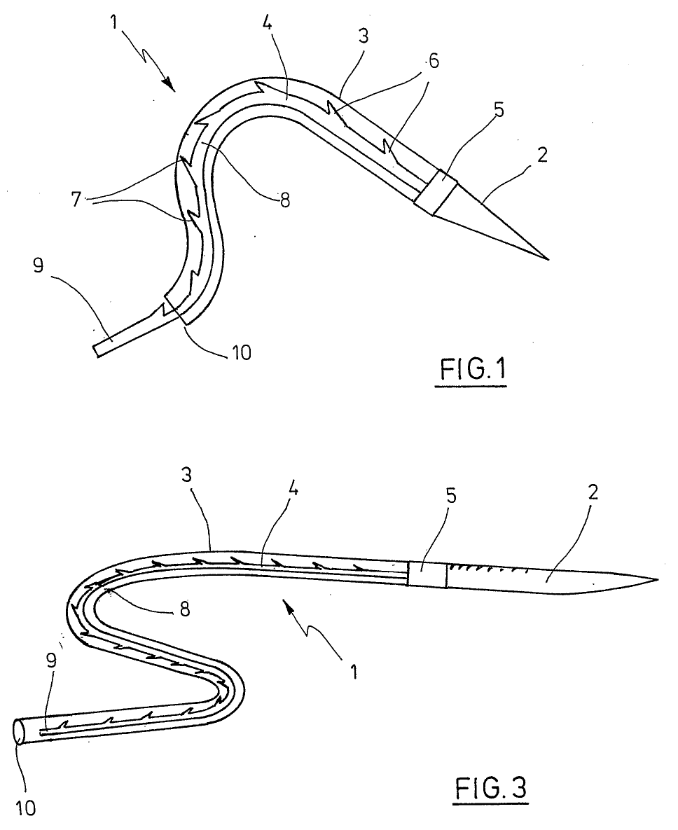

FIG. 1 shows a first illustrative embodiment of a suture system according to the invention,

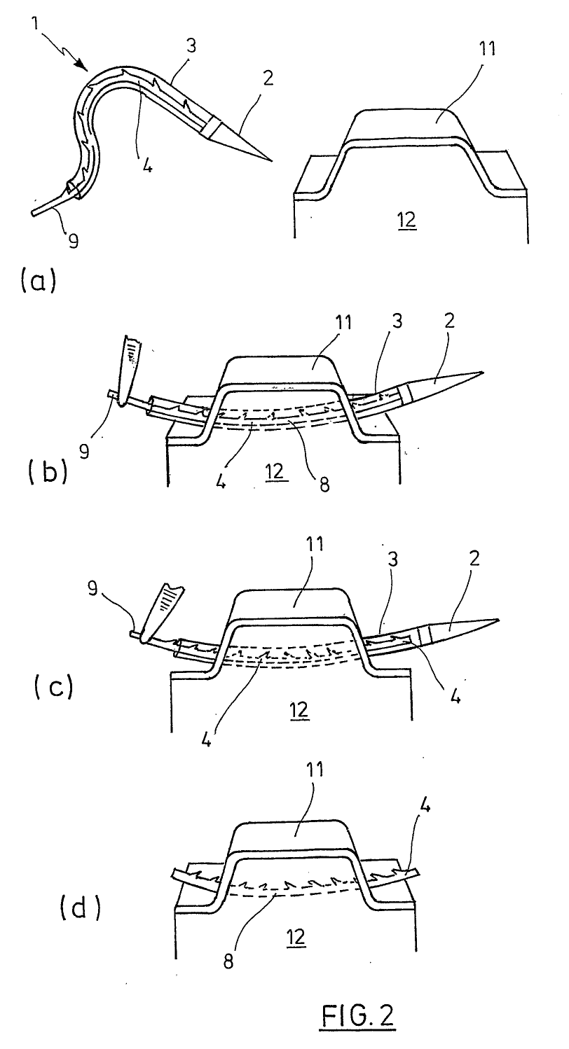

FIG. 2 shows the introduction of the suture system according to the first illustrative embodiment into body tissue,

FIG. 3 shows a variant of the first illustrative embodiment,

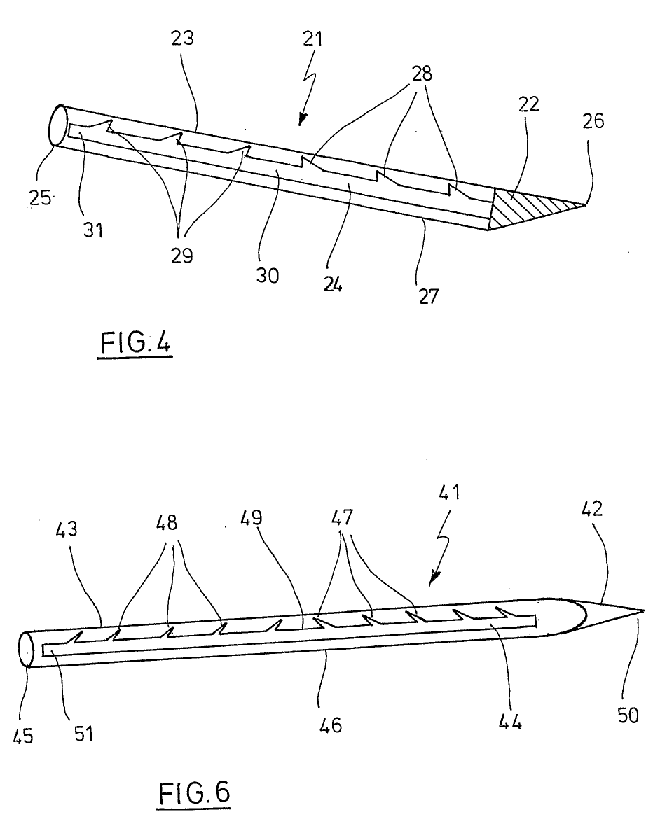

FIG. 4 shows a second illustrative embodiment of a suture system according to the invention,

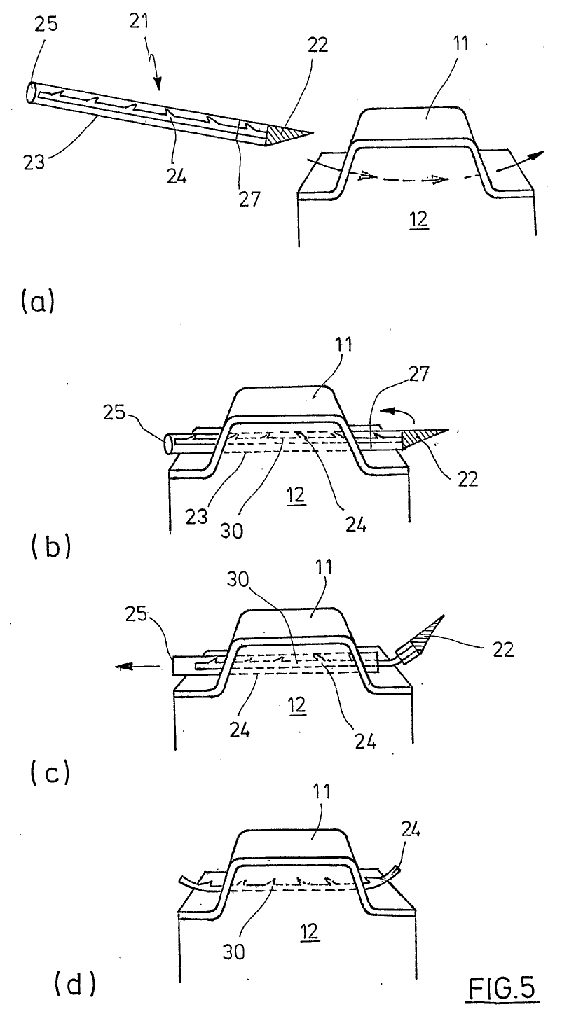

FIG. 5 shows the introduction of the suture system according to the second illustrative embodiment into body tissue, and

FIG. 6 shows a third illustrative embodiment of a suture system according to the invention.

In FIG. 1, a first illustrative embodiment of a suture system 1 according to the invention is shown. The suture system 1 comprises an applicator 2, a sheath 3 and a suture material 4. The applicator 2 is designed as a straight surgical needle which is 120 mm long and has a diameter of 1.2 mm. The needle is provided at the front end with a pointed cutting plate. Alternatively, the tip can also be of a blunt design or can be provided with an asymmetrical cutting edge. Moreover, the tip of the applicator 2 is closed. At the rear end of the applicator 2 there is a rear portion 5 whose external diameter is smaller than the rest of the applicator 2. The rear end of the applicator 2 also has a 3-mm deep bore (not shown) in the axial direction, in which bore the suture material 4 is secured. The suture material 4 is in this case secured using what is known as the controlled-release technique in which the suture material 4 separates from the applicator 2 when a force greater than approximately 6 N is exerted on the suture material 4. In this way, the suture material 4 is connected to the applicator 2 via a predetermined weak point.

In this first preferred illustrative embodiment, the suture material 4 is a 100-mm long Prolene suture material with a multiplicity of first and second barbs 6, 7 cut into it. In this illustrative embodiment, the barbs 6, 7 are arranged at a spacing of 1.2 mm. The first barbs 6, which are arranged on one side of a reference point 8 in the suture material 4, point in a first direction of action oriented towards the reference point 8. The second barbs 7, which are arranged on the other side of the reference point 8, point in a second direction of action counter to the first direction of action and likewise oriented towards the reference point 8. This arrangement of the barbs 6, 7 has the effect that body tissue through which the suture material 4 is drawn is blocked against movement away from the reference point 8. In this first preferred illustrative embodiment, the reference point 8 is arranged at the middle of the suture material 4.

Both the front end area (not shown) and the rear end area 9 of the suture material 4 are free from barbs, in order, on the one hand, to facilitate securing in the applicator 2 and, on the other hand, to facilitate handling of the rear end area 9 of the suture material 4.

As regards the geometry of the barbs 6, 7, the cutting angle and cutting length can be varied so that the height by which the barbs protrude from the main body of the suture material 4 can be adapted to the specific requirements of the body tissue.

The suture material 4 is preferably provided with an active substance, comprising at least one of the substances chosen from the following list: analgesics, paracetamol, local anaesthetics, lidocaine, antibacterial substances, silver, triclosan, fungicides, antiviral substances, anti-inflammatory substances, growth factors, TGF protein, RGDF peptide, vitamin D3, derivatives of vitamin D3, steroids, vitamin A, derivatives of vitamin A, growth-promoting substances, substances which promote healing. In this way, infections are avoided, and the uptake of the suture material 4 in the tissue is improved.

In this preferred illustrative embodiment, the sheath 3 is made of a flexible material such as polyethylene and has an internal diameter of 0.8 mm and a wall thickness of 0.1 mm. Moreover, the sheath 3 is pushed with its front end onto the rear portion 5 of the applicator 2 and is fixedly connected to the applicator by thermal shrinkage. The rear portion 5 of reduced diameter of the applicator 2 ensures that, at the transition between applicator 2 and sheath 3, there is no edge that could cause additional damage during insertion of the suture system 1 into the body tissue. Thus, the transition area between applicator 2 and sheath 3 is of an atraumatic design. The rear end 10 of the sheath 3 is open, such that the suture material 4 emerges at the rear end 10, and the rear end area 9 not provided with barbs remains accessible. The part of the suture material 4 provided with barbs 6, 7 is, however, completely enclosed by the sheath 3. Markings can preferably be provided on the sheath 3 and/or on the applicator 2 in order to indicate the reference point 8, so that the surgeon is able to position the reference point 8, and thus the critical area, in an optimal manner. The applicator 2 and/or the sheath 3 can also contain one of the active substances already mentioned in conjunction with the suture material 4. In addition, the applicator 2 and the sheath 3 can be provided with a lubricant. In this way, it is possible to ensure that damage in the body tissue is as minor as possible and that efficient anchoring of the suture material 4 is achieved.

FIGS. 2a to 2d show the introduction of the suture system 1 according to the first illustrative embodiment through a patient's skin. By means of its tip, the applicator 2 of the suture system 1 is guided through the skin 11 and into the body tissue 12 and then back out of the body tissue 12 through the skin 11, the suture system 1 following a course predetermined by the surgeon. If so required, punctures can be made in the skin 11 at the point of entry and/or point of emergence of the applicator. The applicator 2 designed as a surgical needle is used to cut a path through the skin 11 and body tissue 12 (FIG. 2a).

As a result of the sheath 3, the barbs 6, 7 do not initially come into contact with the body tissue 12, so that the suture system 1 and thus the suture material 4 can be positioned in the desired manner. In particular, the “critical area” around the reference point 8 can be arranged such that it is not disposed near the skin 11 and effective anchoring is ensured.

The rear end area 9 of the suture material 4 not surrounded by the sheath 3 is then held secure (FIG. 2b) and the applicator 2 is drawn in the direction of insertion, so that the suture material 4 separates from the applicator 2 at the separation point formed by the controlled-release connection (FIG. 2c). As the sheath 3 is connected fixedly to the applicator 2, the sheath 3 is drawn out of the body tissue 12 together with the applicator 2, and the barbs 6, 7 of the suture material 4 are freed (FIG. 2d). The barbs 6, 7 then have the effect that the skin 11 and the body tissue 12 can no longer move away from the reference point 8.

FIG. 3 shows a first alternative to the first illustrative embodiment, and, for the sake of clarity, the same reference numbers are used as in FIGS. 1 and 2. Here, the suture material 4 can be fixedly connected to the applicator 2, whereas the sheath 3 is fitted on the applicator with a controlled-release join as the separation point. In this alternative too, the rear end 10 of the sheath 3 extends beyond the rear end area 9 of the suture material 4 and is closed. In such a case, the introduction of the suture system 1 into the body tissue 12 first takes place as in FIG. 2a. Thereafter, however, the applicator 2 is held secure and the sheath is drawn back counter to the direction of insertion and the suture material 4 is released. Finally, the applicator 2 can be detached from the suture material 4.

According to a second alternative to the first illustrative embodiment, the rear end of the suture system 1 can be provided with a further applicator. In this case, the front applicator is then connected to the suture material by a controlled-release connection, and the sheath is connected fixedly to the front applicator. By contrast, the second rear applicator is fixedly connected to the suture material, while the sheath is fitted on the applicator by a controlled-release connection. For identification purposes, the two applicators are differently marked in order to show on which applicator the suture material is fixedly mounted and on which applicator it is releasably mounted. A suture system according to this second alternative is introduced into the body tissue by a procedure in which the front applicator is first guided through the tissue by means of its tip (optionally also by a puncture section) and the rear applicator is then also guided through the body tissue, starting from the same location. The directions in which the two applicators are guided do not generally form a straight line. The two applicators are then drawn in the opposite direction so that the sheath is removed from the body tissue and the suture material is released.

In the second alternative, it is also possible that the suture material is not connected at all to the second applicator.

FIG. 4 shows a second illustrative embodiment of a suture system 21 according to the invention. The second illustrative embodiment likewise comprises an applicator 22, a sheath 23 and a suture material 24.

The applicator 22 is designed as a straight surgical needle on which the suture material 24 is fixedly arranged, and the applicator 22, as in the first illustrative embodiment, has an axial bore (not shown) at its rear end. A tubular body measuring 90 mm in length serves as sheath 23, and the rear end 25 of the sheath 23 in this illustrative embodiment is closed and the point of the sheath 23 is open. The sheath 23 is made of a rigid material such as stainless steel, which is preferably bendable.

The applicator 22 is inserted into the open tip of the sheath 23 and connected fixedly to the sheath 23, such that said sheath 23 starts with its front area from the rear end area of the applicator 22 and surrounds the suture material 24. At its rear area, the applicator 22 has a stepped area of reduced diameter (not shown) which is fitted into the open tip of the sheath 23. In this way, the transition area between applicator 22 and sheath 23 has no edges or projections and is thus also of an atraumatic design. Provided at a distance of 4 mm from the front end of the sheath 23 there is a separation point which is designed as a predetermined weak point 27 extending perpendicular to the longitudinal axis of the suture system 21.

As in the first illustrative embodiment, the suture material 24 is preferably a Prolene suture material provided with a multiplicity of first and second barbs 28, 29. The first barbs 28, which are arranged on one side of a reference point 30 in the suture material 24, point in a first direction of action oriented towards the reference point 30. The second barbs 29, which are arranged on the other side of the reference point 30, point in a second direction of action likewise oriented towards the reference point 30. This arrangement of the barbs 28, 29 has the effect that the body tissue is blocked against movement away from the reference point 30. Here too, for better handling, the front end area and the rear end area 31 of the suture material 24 are not provided with barbs.

FIGS. 5a to 5d show the introduction of the suture system 21 according to the second illustrative embodiment into body tissue. First, the suture system 21 is guided through the skin 11 into the body tissue 12, and, because of the stability of the sheath 23, the suture system 21 can also be guided from its rear end 25. The suture system 21 is pushed through the body tissue 12 until the predetermined weak point 27 again emerges from the body tissue 12 and is accessible (FIGS. 5a and 5b). The suture system 21 can be readily moved inside the body tissue 12 until the desired position, in particular of the reference point 30, is reached. The sheath 23 is then separated at the separation point 27 and can be drawn out of the body tissue 12 counter to the direction of insertion, so that the barbs 28, 29 are freed (FIG. 5c). Finally, the applicator 22 and the part of the sheath 23 remaining on it are separated from the suture material 24, so that only the suture material 24 remains in the body tissue 12 and blocks the latter against movement away from the reference point 30 (FIG. 5d).

In an alternative to the second illustrative embodiment, the suture material 24 may also not be secured on the applicator 22 and may instead be arranged loosely inside the sheath 23. After insertion of such a suture system 21 into the body tissue 12, in this alternative the sheath 23 is again broken open along the predetermined weak point 27 and the applicator 22 and the front part of the sheath 23 are removed, such that the free front part of the suture material 24 can be held secure as the sheath 23 is pulled off backward.

FIG. 6 shows a third preferred illustrative embodiment of a suture system 41 according to the present invention. The suture system 41 comprises an applicator 42, a sheath 43 and a suture material 44, the applicator 42 and sheath 43 being made in one piece. The sheath 43 has its front end area starting from the rear end area of the applicator 42 and surrounds the suture material 44, the rear end 45 of the sheath 43 being closed. The sheath 43 has a separation point which is designed as a predetermined weak point 46 and which, with respect to the front end of the suture material 44, is offset towards the rear end 45 of the sheath 43.

The suture material 44 is provided with first and second barbs 47, 48 which are arranged relative to a reference point 49 in the same way as in the first and second illustrative embodiments. The suture material 44 is not secured on the applicator 42, but instead arranged loosely in the sheath 43.

When introducing the suture system 41 according to the third illustrative embodiment, the applicator 42, made in one piece with the sheath 43, is pushed through the body tissue so that the predetermined weak point 46 is again freely accessible. The sheath 43 is then broken open at the predetermined weak point 46 and the part with the applicator 42 is removed. Since the predetermined weak point 46 is offset towards the rear end 45 of the sheath 43, a sufficiently large part of the suture material 44 is then exposed, so that the suture material 44 can be held directly, while the remaining part of the sheath 43 is drawn from the body tissue via its rear end 45.

In all the illustrative embodiments shown, the tip (26, 50) of the applicator (2, 22, 42) can be surrounded by a retractable safety sleeve, by which means the tip (26, 50) is protected so that the risk of injury to the surgeon is reduced.

In the illustrative embodiments shown, the course of the suture material can be easily corrected by moving the suture material with the sheath backwards and forwards, without causing further damage to the tissue. Without the sheath, the barbs would tear fibres when moved in the direction in which the barbs are pointing. In particular, the barbs do not “saw” into the tissue.

Moreover, the suture system according to the invention can be introduced directly by the surgeon without any need for preparatory measures. This saves time and reduces the risk of infection.

1. Surgical suture system with
a needle-like applicator (2, 22, 42) having a tip (26, 50) in its front end area,
a surgical suture material (4, 24, 44) provided with barbs (6, 7, 28, 29, 47, 48), and
a sheath (3, 23, 43) whose front end area starts from the rear end area (5) of the applicator (2, 22, 42) and which surrounds the surgical suture material (4, 24, 44).

2. Surgical suture system according to claim 1, characterized in that, on one side, the barbs (6, 7, 28, 29, 47, 48) point in one direction of action from a reference point (8, 30, 49) on the suture material (4, 24, 44) and, on the other side, point in the opposite direction of action from the reference point (8, 30, 49) and are designed to block a movement of body tissue (12) away from the reference point (8, 30, 49).

3. Surgical suture system according to claim 1 or 2, characterized in that the sheath (3, 23, 43) comprises a flexible material.

4. Surgical suture system according to claim 1 or 2, characterized in that the sheath (3, 23, 43) comprises a rigid material which is optionally bendable.

5. Surgical suture system according to claim 4, characterized in that the applicator (2, 22, 42) and the sheath (3, 23, 43) are made in one piece.

6. Surgical suture system according to one of claims 1 to 5, characterized in that a separation point is provided in the zone reaching from the rear end area (5) of the applicator (2, 22, 42) to the front end area of the sheath (3, 23, 43).

7. Surgical suture system according to claim 6, characterized in that the separation point has a predetermined weak point (27, 46).

8. Surgical suture system according to claim 6, characterized in that the sheath (3, 23, 43) can be detached from the applicator (2, 22, 42) at the separation point.

9. Surgical suture system according to one of claims 1 to 8, characterized in that the suture material (4, 24, 44) is secured on the applicator (2, 22, 42).

10. Surgical suture system according to claim 9, characterized in that the suture material (4, 24, 44) is secured on the applicator (2, 22, 42) via a predetermined weak point.

11. Surgical suture system according claim 10, characterized in that the sheath (3, 23, 43) is open at its rear end (10, 25, 45), and the suture material (4, 24, 44) protrudes from the rear end (10, 25, 45) of the sheath (3, 23, 43).

12. Surgical suture system according to one of claims 1 to 8, characterized in that the suture material (4, 24, 44) is not secured on the applicator (2, 22, 42).

13. Surgical suture system according to claim 12, characterized in that the sheath (3, 23, 43) has a separation point which, in relation to the front end of the suture material (4, 24, 44), is offset towards the rear end (10, 25, 45) of the sheath (3, 23, 43).

14. Surgical suture system according to one of claims 1 to 10, 12 or 13, characterized in that the sheath (3, 23, 43) is closed at its rear end (10, 25, 45).

15. Surgical suture system according to one of claims 1 to 13, characterized in that the sheath (3, 23, 43) is open at its rear end (10, 25, 45).

16. Surgical suture system according to one of claims 1 to 15, characterized in that the transition area between applicator (2, 22, 42) and sheath (3, 23, 43) has an atraumatic design.

17. Surgical suture system according to one of claims 1 to 16, characterized in that the applicator (2, 22, 42) comprises a surgical needle.

18. Surgical suture system according to one of claims 1 to 17, characterized in that the tip (26, 50) of the applicator (2, 22, 42) is surrounded by a retractable safety sleeve.

19. Surgical suture system according to one of claims 1 to 18, characterized in that the tip (26, 50) of the applicator (2, 22, 42) is closed.

20. Surgical suture system according to one of claims 1 to 19, characterized in that the tip (26, 50) of the applicator (2, 22, 42) has one of the configurations chosen from the following list: blunt, provided with asymmetrical cutting edge, provided with symmetrical cutting edge.

21. Surgical suture system according to one of claims 1 to 20 in conjunction with claim 2, characterized in that the reference point (8, 30, 49) lies in the middle area of the suture material (4, 24, 44).

22. Surgical suture system according to one of claims 1 to 21, characterized in that the front end area of the suture material (4, 24, 44) and/or the rear end area of the suture material (9, 31, 51) is/are free of barbs.

23. Surgical suture system according to one of claims 1 to 22, characterized in that the suture material (4, 24, 44) is provided with an active substance.

24. Surgical suture system according to claim 23, characterized in that the active substance comprises at least one of the substances chosen from the following list: analgesics, paracetamol, local anaesthetics, lidocaine, antibacterial substances, silver, triclosan, fungicides, antiviral substances, anti-inflammatory substances, growth factors, TGF protein, RGDF peptide, vitamin D3, derivatives of vitamin D3, steroids, vitamin A, derivatives of vitamin A, growth-promoting substances, substances which promote healing.

25. Surgical suture system according to one of claims 1 to 24 in conjunction with claim 2, characterized in that the sheath (3, 23, 43) and/or the applicator (2, 22, 42) is/are provided with markings to indicate the reference point (8, 30, 49).

26. Surgical suture system according to one of claims 1 to 25, characterized in that the sheath (3, 23, 43) and/or the applicator (2, 22, 42) has/have a lubricant.

27. Surgical suture system according to one of claims 1 to 26, characterized in that the sheath (3, 23, 43) and/or the applicator (2, 22, 42) comprises an active substance, preferably at least one of the substances chosen from the following list: analgesics, paracetamol, local anaesthetics, lidocaine, antibacterial substances, silver, triclosan, fungicides, antiviral substances, anti-inflammatory substances, growth factors, TGF protein, RGDF peptide, vitamin D3, derivatives of vitamin D3, steroids, vitamin A, derivatives of vitamin A, growth-promoting substances, substances which promote healing.

28. Surgical suture system according to one of claims 1 to 10, 12, 13 or 16 to 27, characterized in that a further applicator is arranged at the rear end (10, 25, 45) of the sheath (3, 23, 43).

29. Surgical suture system according to claim 28, characterized in that the two applicators are differently marked.
